(19) United States
(12) Patent Application Publication
(10) Pub. No.: US 2023/0256004 A1
(43) Pub. Date: Aug. 17, 2023

(54) COMPOSITIONS AND METHODS FOR TREATING MOOD DISORDERS AND CIRCADIAN RHYTHM SLEEP DISORDERS

(71) Applicant: Light Aqua LLC, Albuquerque, NM (US)

(72) Inventor: William Ulwelling, Albuquerque, NM (US)

(73) Assignee: Light Aqua LLC, Albuquerque, NM (US)

(21) Appl. No.: 18/015,011

(22) PCT Filed: Jul. 8, 2021
(86) PCT No.: PCT/US2021/040936

§ 371 Date: Jan. 6, 2023

Related U.S. Application Data
(60) Provisional application No. 63/049,844, filed on Jul. 9, 2020.

(51) Int. Cl. A61K 33/00; A61K 31/5386

(52) U.S. Cl. CPC A61K 33/00; A61K 31/5386

(57) ABSTRACT

Compositions and methods for treating depression, anxiety and/or circadian rhythm sleep disorders using deuterium oxide and various compounds thereof.

### CROSS-REFERENCE TO RELATED APPLICATIONS

This application claims priority to and the benefit of the filing of U.S. Provisional Pat. Application No. 63/049,844, entitled “Compositions and Methods for Treating Mood Disorders and Circadian Rhythm Sleep Disorders”, filed on Jul. 9, 2020, and the specification and claims thereof are incorporated herein by reference.

### BACKGROUND OF THE INVENTION

### Field of Invention

An embodiment of the present invention relates in general to medications for treating psychiatric disorders and in particular to the use of Deuterium Oxide for treating mood disorders (depression or anxiety) and circadian rhythm sleep disorders.

### Background Art

Numerous treatments for mood disorders (as defined in the Diagnostic and Statistical Manual of Mental Disorders, Fifth Edition, of the American Psychiatric Association) have been developed. Antidepressant medications are one of the most commonly used treatments for depression. While often successful in treating depression, they have a number of shortcomings. They generally take two weeks or more to take effect, a delay associated with significant suffering, lost productivity, treatment cost, morbidity, and occasional fatalities. A significant minority of sufferers are not helped by any of the available antidepressants. The side effects of current antidepressants are problematic to many, often so much so that the medication becomes intolerable and needs to be discontinued. Some antidepressant medications can lead to abuse or addiction. Even if an antidepressant is well tolerated and will eventually be effective for the patient, it is currently difficult to dose the medicine precisely in order to obtain a therapeutic serum level in the patient’s body, particularly in the brain. Wide variability in individual rates of absorption and metabolism of the current antidepressants make it difficult to estimate the concentration of the medicine in the blood. Blood sampling, an invasive procedure troublesome to most patients, is required for an accurate determination of whether a therapeutic serum level has been obtained.

Other physical treatments for depression, based on chronobiological theories of depression and mood disorders described below, have been used successfully to treat depression. Circadian rhythms are commonly disordered in depressed subjects, and the derangements of human biological rhythms are an integral part of a depressive disorder. A number of therapeutic manipulations of circadian rhythms have successfully treated depression, but these treatments have numerous shortcomings as well. Sleep deprivation therapy--awakening the patient for the day at 4 a.m. or earlierreliably produces a rapid antidepressant effect that day. Its rapid onset thus overcomes the long delays in treatment response associated with antidepressant medications; however, the antidepressant effect of sleep deprivation therapy lasts only till the next normal night’s sleep. Light therapy is effective for seasonal affective disorders (a sub-type of depression which recurs on a seasonal basis), but these treatments are inconvenient and impractical compared to medication treatments.

Deuterium oxide was the first substance found to change the circadian rest/activity cycle in animals, lengthening the period of this cycle in animals drinking it. Deuterium is a stable, naturally-occurring isotope of hydrogen, and when combined with oxygen forms deuterium oxide, or “heavy water.” It diffuses evenly and rapidly throughout the total body water and thus is a good label to measure total body water in humans and other animals. It has been given safely to nursing mothers and babies in studies evaluating effectiveness of nursing. It has also been given safely to children and adults in numerous other scientific studies. It has never been used, however, for the purpose of treating depression or any mood disorder in humans.

The “master clock” which regulates most--perhaps all--of the various biological rhythms in humans is located in the suprachiasmatic nuclei structure of the brain. Very few substances (including very few medications) are capable of disturbing this clock, and one of the first found to do so was deuterium oxide. Since then, almost all antidepressant medications tested have also been shown to possess the ability to alter circadian rhythms in animals. To date, however, no one has administered or suggested administering deuterium oxide to humans for the treatment of depression.

Circadian Rhythm Sleep Disorders, as defined in DSM-5, involve a mismatch between the individual’s endogenous circadian sleep/wake rhythms and exogenous demands regarding the timing and duration of sleep. “Jet Lag” type and “Shift Work” type are two types of this disorder. Various behavioral manipulations of the sleep/wake cycle have been tried—with limited success--to treat these disorders. Benzodiazepines alter circadian rhythms and have been used to treat these disorders, but these agents have a high abuse potential, and can cause side effects of excess sedation, and amnesia. Melatonin alters circadian rhythms and has been used to treat these disorders, but sometimes causes side effects of sedation, and is ineffective for many people. Deuterium oxide is a substance which can directly alter the sleep/wake cycle. Therapeutic use of this property, however, has not yet been used to treat these two disorders.

Nothing in the art suggests using water saturated with the proper amount of deuterium oxide to treat depression and anxiety. The opposite was suggested in U.S. Pat. No. 6,872,408 (“Bell”), which suggested using isotopically pure hydrogen-containing water substantially free of deuterium as a treatment for bipolar depression, which is a particular sub-type of depression that occurs in the context of bipolar disorder - formerly called “manic-depressive illness”. Bell does not describe use of water substantially saturated with deuterium to treat depression or anxiety. Rather, Bell describes the use of water substantially saturated with deuterium to treat mania. However, mania is a medically distinct condition from depression. In depression, the mood is abnormally depressed, while in mania the opposite occurs, and the mood is abnormally elevated. What is needed is a deuterium oxide compound and/or mixture and/or solution and/or composition capable of treating depression and anxiety, as proposed by an embodiment of the present invention.

The related art also includes U.S. Pat. No. 5,223,269 (“Liepins”), for use of heavy water as antihypertensive medication. Liepins’ theoretical underpinning includes animal studies showing that ingested heavy water reduces blood pressure in animals. Liepins addresses hypertension, but does not describe the use of deuterium oxide for any psychiatric disorder, including depression or anxiety. Liepens specifies the use of very low dosages of deuterium oxide for treatment of hypertension specifically, which would be ineffective for treatment of depression. What is needed is a compound of heavy water (deuterium oxide) and/or composition comprising deuterium oxide in higher dosages capable of penetrating the brain’s suprachiasmatic nucleus and affecting the circadian rhythms it controls and synchronizes, as proposed by an embodiment of the present invention.

### 1. Defining “Mood”

In order to understand the medical underpinnings of one or mor embodiments of the present invention, a workable definition of the word “mood” should be understood. One way to define the vague concept of “mood” is as a predisposition for a particular type or level of activity or inactivity. This definition of mood evokes an evolutionary history. The internal ability of an organism to predispose itself for response to a regularly changing environment will effectively anticipate the recurrent change and add immensely to fitness. Circadian predispositions adapted organisms to life on a planet rotating on its axis and revolving about the sun. Rather than passively respond to day and night environments, an internal clock allowed anticipation of these regular changes with an appropriate predisposition. Developing such circadian rhythms is a capability within all cells; for example, cyanobacteria acquired it over 2 billion years ago. At higher levels of organization, organisms’ circadian clocks became more complex to include circannual rhythms. In addition to circadian rhythms, seasonal rhythms were added to the repertoire of the internal clocks. Come winter, bears’ internal clocks signal it’s time for a predisposition for a particular sort of activity, hibernation. In late fall, the bear develops an “increased appetite” and gains weight, and by early winter the bear shows “less interest in activities,” “manifests reduced energy,” and begins “sleeping too much.” (See DSM-5 diagnostic criteria for a particular Mood Disorder, Seasonal Affective Disorder). In addition to circa-annual rhythms, further capabilities evolved to give the organism the ability to internally set predispositions for particular types of activity and inactivity. These capabilities are the evolutionary anlagen of “mood.” This evolutionary perspective on mood, properly defined, yields one theoretical explanation of why manipulations of the body’s clock affects mood.

A functional definition of mood highlights the significance of circadian rhythms in the evolutionary history of mood. Mood disorders in humans are highly correlated with abnormal and desynchronized circadian rhythms of neuroendocrine and sleep/wake subsystems. Two validated and replicated chronobiological treatments-light therapy and early-awakening therapy-suggest that chronobiological interventions are correcting an underlying cause of depression. One characteristic of these treatments is that when they do work, they work quickly, suggesting a direct action upon a primary etiological factor. The “master clock” that controls and synchronizes mammals’ circadian rhythms is the Suprachiasmatic Nucleus in the brain. The first substance known to penetrate the SCN and alter circadian rhythms was deuterium oxide (“heavy water”). Heavy water is an ideal chronopharmaceutical in that the deuterium isotope’s chemical properties differ from the main hydrogen isotope almost solely in the kinetics of its reactions. Over the past five decades, the biological effects of heavy water have been thoroughly studied, including a dose-response relationship for altering circadian rhythms in mammals. Safety thresholds have been proposed for numerous species, including humans. Ingestion of measured amounts of heavy water by human subjects has been done in numerous monitored trials. Heavy water is commonly given to infants and nursing mothers in hydration studies. An acute dose high enough to resynchronize and reset human circadian rhythms is approximately 15 times less than the dose considered a safe threshold for chronic heavy water ingestion. A falsifiable hypothesis derived from the chronobiological theory outlined above is that heavy water will be a safe, efficacious, rapidly-acting, non-addictive, low-side-effect antidepressant medication.

### 2. Disorders of Mood and Circadian Rhythms

Since the time of Hippocrates, physicians noticed that the characteristic symptoms of depressive disorder changed according to a circadian pattern (e.g., sleep disturbance characterized by early morning wakening, and a diurnal worsening in “melancholia”). Mood disorders are typically episodic and recurrent, sometimes recurring with a seasonal pattern. When intermittent manic states are added, in Bipolar Disorder, the chronobiological factors become even more prominent. Major Depressive Disorders (MDD) are correlated with disruptions of the circadian rhythms in sleep, activity, REM sleep latency, temperature, appetite and digestion, immune function, cortisol, thyroid-stimulating hormone, norepinephrine, melatonin, prolactin, and growth hormone. Most typically, circadian rhythms in depressive patients are desynchronized. MDD seems to be related to a disruption in the master circadian clock function and not to an alteration in a specific rhythm. These disrupted rhythms generally return to normal when the depression lifts.

The DSM-5 diagnostic criteria of Major Depressive Disorder describe a chronobiologic syndrome hiding in plain sight. Over the past decades, chronobiologists’ studies have focused on a few particular behaviors that have a notable circadian pattern: feeding, sleeping, being wakeful, and being active. These four terms also are used to describe the four so-called “vegetative” signs of depression (see DSM-5 MDD diagnostic criteria #3,4,5,6). Circadian control of levels of cognitive alertness arguably links it to MDD criterion #8 (diminished ability to think, concentrate). The so-called “somatic” symptoms of depression include this criterion #8 (as do some definitions of “vegetative” symptoms). The DSM-5 specifier of MDD with “melancholic features” adds the chronobiologic characteristic of early morning wakening, and diurnal variation in severity. The two remaining MDD diagnostic criteria describe emergent symptoms in higer-level psychological systems (guilt/worthlessness, and suicidality). Surprisingly, the diagnosis of Major Depressive Disorder does not require the presence of a “depressed mood” (e.g., “sad, blue”). An experience of “loss of interest” in life will do just as well for example, a patient might describe such a state as: “I don’t feel depressed. I just feel like someone pulled the plug out.”

These correlations between mood disorders and chronobiological abnormalities are intriguing, but they don’t necessarily imply that chronobiological upset is an underlying pathological mechanism for mood disorder. However, when chronobiological interventions cure the depression, then a causal role is more strongly suggested. There are at least two proven, frequently-replicated chronobiological treatments for MDD: bright light therapy, and wake therapy. In Wake Therapy, the depressed subject is wakened around 3am and kept awake through the following day. The depression remits in 60% of patients. The intervention has many problems, most notably the fact that the cure is short-lived, and depression returns after the first night of uninterrupted sleep. Bright Light Therapy proponents theorize a mechanism of action whereby a bright light stimulus corrects internal misalignment of circadian rhythms. One common characteristic of these two chronotherapeutic measures is especially significant: when they work, they take effect very rapidly.

More speculatively, a third rapidly acting treatment of mood disorders may have a mechanism of action involving its action on the master clock in the suprachiasmatic nucleus (SCN). Electroconvulsive Therapy (ECT) not infrequently takes full effect after only one treatment, and half the total decline in HAM-D scores generally occurs after the first treatment. ECT does correct the abnormal circadian rhythms in depressives, and ECT alters the expression and daily oscillation of circadian genes in the frontal cortex of rats. In ECT, it is the seizure-not the electricity-that is the curative factor, and chemically-induced seizures are just as effective. A generalized grand-mal seizure may well be the zeitgeber that resets/resynchs the neurons of the SCN, as it does the neurons in the rest of the brain.

It is possible that the mechanism of action of other established treatments for mood disorders may involve their chronobiological effects, their effect on the SCN “clock.” Lithium, the gold standard for treatment of Bipolar Disorder, lengthens the period of SCN neurons, through its inhibition of the glycogen synthetase kinase (GSK-3b) enzyme important to the function of the Transcription-Translation Feedback Loop mechanism of the intracellular clock. Since the SCN has receptors for monoamines, and neural connections to monoaminergic centers, it is affected to some degree by neurotransmitter-based antidepressants as well.

Melatonin or structurally-similar agents are chronobiologically-based pharmaceutical agents that are FDA-approved as hypnotics (e.g., Ramelteon, Tasimelton), or are approved as an augmenting medication for treating depression (e.g., Agomelatine). The SCN controls the secretion of melatonin from the pineal gland, and a melatonin negative-feedback-loop signal is received by receptors on the SCN. Melatonin affects the phase position of the sleep-wake cycle and is a proven hypnotic, but is not an effective antidepressant. The antidepressant Agomelatine affects the 5-HT 2c histamine receptor as well as being a long-acting agonist at the M1 melatonin receptor on the SCN. Its mechanism of action may involve chronobiologic as well as traditional antidepressant serotonergic effects. The chronobiologic effect may be primarily due to a melatonergic improvement of sleep. The 2+ week time-of-onset of Agomelatine suggests a serotonin-reuptake-inhibition mechanism of action through an initial neurotransmitter reuptake inhibition followed by a series of adjustments in individual neurons and neuronal networks.

The primary chronobiological circadian rhythm abnormality in MDD is generally believed to be a desynchronization of normal or impaired circadian rhythms directed by the SCN clock. The SCN is the only nucleus in the brain that can generate and sustain circadian gene expression and physiological rhythms without any outside influence. One interesting characteristic of the SCN clock is that it is “designed” to be reset each day. The period of the SCN clock is 24 h 11 min, about 1% longer than the 24 h 0 min period of the earth’s rotation. The light of every day’s sunrise resets the SCN clock for that day, which always has a slightly different photoperiod than the day before. Light is a major “zeitgeber” or “time-giver” to the SCN clock, giving it a new “time zero” reset for each new day. Other zeitgebers include darkness (signaled by melatonin), and activity/rest level. Humans have become particularly sensitive to behavioral, social inputs as zeitgebers. A clock in the liver responds best to a feeding zeitgeber. The SCN can also detect and react to seasonal rhythms. In mammals the SCN is necessary to respond to the changes in length-of-day that announce a new season. Circadian rhythms are generated within each SCN cell, and sustained and synchronized by diverse cell-cell interactions. Solving the genetic-molecular mechanism of the 24-hour timing system within cells of Drosophila earned its discoverers the 2017 Nobel Prize in Physiology and Medicine. The 10,000 neurons of each of the two bilateral human SCN’s internally communicate using various neurotransmitters. The neurons internally synchronize, and then send out signals through other neural connections and hormonal outputs to synchronize the numerous “slave clocks” throughout the body. Also, the SCN projects monosynaptically to multiple hypothalamic nuclei that subsequently communicate with regions that synthesize dopamine, serotonin, and norepinephrine. Resetting the clock network involves circuit-wide readjustments within the SCN and the cells to which it signals. Initial theories about the nature of the circadian abnormality that might underly a depressive disorder focused on an out-of-phase (phase-advanced) sleep/activity cycle. Now, the consensus theory favors a desynchronization of the SCN clock, internally and/or with its slave clocks.

The two successful chronobiological treatments (three, if you count ECT) putatively act by causing a reset/resynch of a desynchronized clock. SAD treatments use bright light to reset/resynch; Sleep-deprivation/Wake-Therapy treatments use an activity/sleep-disruption to reset/resynch; ECT uses a generalized tonic-clonic seizure to reset/resynch. What is needed is a pharmaceutical reset/resynch of the clock by administering a pulse of deuterium oxide, as proposed by embodiments of the present invention.

### 3. Deuterium Oxide

Deuterium Oxide was one of the earliest research tools in chronobiology, and one of the first substances known to penetrate into the SCN, and alter circadian rhythms. Deuterium Oxide (“heavy water”) is a water molecule in which the most common hydrogen isotope (protium) is replaced with the less-common isotope (deuterium). Protium is made up of a nucleus of one proton and one orbiting electron. Deuterium has one electron in its electron shell, but the nucleus of the deuterium isotope contains one proton and one neutron. Since the number of negatively charged electrons in outer electron shell is the same for both protium and deuterium, their chemical reactions are basically the same. Heavy water participates in the same chemical reactions as regular water and penetrates into the body everywhere that water goes.

Deuterium oxide is chemically equivalent to regular water, but due to a kinetic isotope effect, these reactions are slowed. In deuterium oxide, the deuterium-oxygen bond is stronger than a protium-oxygen bond and is harder/slower to break. Also, a hydrogen bond between adjacent hydrogen-containing molecules is stronger with the deuterium isotope of hydrogen.

When the protium isotope of hydrogen is replaced by deuterium, a kinetic isotope effect of at least two- to three-fold occurs to slow the chemical reactions. If deuterium replaces protium in a carbon-hydrogen bond, subsequent cleavage of this bond slows by a factor as high as 9-fold. This kinetic isotope effect is particularly large for isotopes of hydrogen, since the one additional neutron in deuterium approximately doubles the nuclear mass of protium. Introducing a significant amount of deuterium oxide into the body will affect all chemical reactions in the body involving water or hydrogen-which is to say, just about all reactions. Particularly sensitive to kinetic changes are the tissues with a purely kinetic function-- the “clocks” within the body. Experiments have shown that the master clock in the SCN shows measurable changes by heavy water before other organ systems show any effect. In 1972, Dowse noted that the “chronornutagenic” effects of 5% deuterium oxide occurred within 24 hours of dosing. At high concentrations, other tissues begin to show effects as well.

The 20,000 cells in the suprachiasmatic nuclei constitute the dominant circadian pacemaker in mammals. The SCN entrains the entire timing network after receiving retinal input directly through the retino-hypothalamic tract. Each SCN cell generates an independent circadian rhythm, as a number of particular genes are transcribed to RNA molecules, which translate particular proteins, which in turn migrate out of the cell nucleus and form dimers that then make their way back into the nucleus and inhibit the initiating genes of this 24-hour feedback loop. The 20,000 neurons within the SCN also synchronize via neuropeptide signaling. The SCN then signals to myriad other centers and clocks throughout the body. The targets of these signals in the body can return hormonal and neurotransmitter signals back to the SCN, and these feedback signals in turn modify the SCN’s signaling. Unlike the peripheral clocks, the SCN will continue its high amplitude molecular and electrical circadian oscillations in vitro.

While this synchronized timekeeping circuit is incredibly complex, one salient fact of deuterium oxide’s action is known: deuterium oxide slows down the clock and the circadian rhythms the clock generates and synchronizes. This significant time-keeping disruption is hypothesized to cause a reset/resynch of the timing system. When we use words like “clock” and “slowing” we are of course speaking the language of kinetics, whose domain is the rate and timing of processes. A clock is a kinetic mechanism, and heavy water is used in this medical invention as a relatively pure kinetic intervention, basically affecting only the rate of the reactions within the clock. Once deuterium oxide penetrates into the SCN master clock, a significant increase in deuterium occurs and we “deuterate the brain’s master clock.” Some or all of the reactions involved in the timing process are significantly slowed. The mechanism of action of this deuteration of particular molecules within the time-keeping feedback loop likely involves some rate-limiting chemical reactions, and some large complex molecules containing many hydrogen (now replaced with deuterium) atoms. Again, the mechanism details are not as important as the fact that a measurable slowing of bodily circadian rhythms occurs within 24 hours.

### 4. Chronobiology Studies Using Deuterium Oxide

The first substance found to penetrate the blood-brain barrier and affect the rest/activity circadian cycle was deuterium oxide. In 1968, Suter and Rawson showed that deuterium oxide lengthened the period of the activity rest cycle in white mice. The period lengthened directly and linearly with the concentration of deuterium oxide, up to a 6% increase in period length with the maximum 30% deuterium oxide dose administered. Dowse’s 1972 study reported the “chronomutagenic effect” of deuterium oxide on mice: a 5% deuterium oxide concentration lengthened the period of circadian rest/activity cycle by 1% (25 minutes). The effect was rapid, taking effect within 24 hours. Richter’s 1977 studies showed that hamsters’ rest/activity clock was freed from entrainment to light when they drank 10% deuterium oxide. The hamsters’ circadian rhythm began to fluctuate when the hamsters drank 5% deuterium oxide, and also in one hamster drinking 1% deuterium oxide. When drinking-water reached 10% deuterium oxide, the hamsters’ rest/activity cycle was freed from entrainment by light/dark inputs. Deuterium oxide has slowed the internal clocks of hamsters, rats, white mice, and squirrel monkeys. Mammals show measurable changes in their circadian rhythms after drinking small amounts of deuterium oxide. Hamsters show a linear response to increasing deuterium oxide dosages, with no lower threshold. Hamster circadian rhythms were slowed on average about 1% for every 5% deuterium oxide concentration increase in their total body water, with slowing also noted in some hamsters given 1% deuterium oxide. In a 1972 review, Peng recommended a 5% deuterium oxide concentration as safe long term for rats, and 1% as safe long term for humans. Compared to mice, humans are roughly 5 times as sensitive to the toxic effects of deuterium oxide. Regarding a dosage to achieve therapeutic effect, it is reasonable to conjecture that humans may be 5 times as sensitive to the chronobiotic effects of deuterium oxide as well. Deuterium oxide has never been tried as an antidepressant or anti-manic agent.

Regarding the efficacious dose of deuterium oxide for therapeutic effect, an important preliminary consideration is that the SCN is a precise, sensitive and well-insulated master clock. The inherent period of the human SCN clock needs to be longer than 24 h to be reset each dawn, but the period of the SCN master-clock is only about 0.8% longer than 24 hours (about 11 minutes). The SCN clock is sensitive enough to detect changes in length of the photoperiod (dawn-dusk) in order to implement seasonal changes, even though the daily photoperiod changes are only about a minute or two per day (depending on latitude). At 40 degrees N latitude (Washington DC, Rome, Beijing) there is about a 1% (about 15 minute) change per week in the daily photoperiod. The human SCN clock regularly resets when about <1% (about 11 min) of its innate period remains, and it can detect changes of about 1% in the photoperiod to trigger seasonal changes. What is needed is a therapeutic intervention capable of altering the clock period by at least about 1% in order to achieve reset/resynch, as the present invention would provide. Deuterium oxide can be delivered safely to humans to achieve concentrations about 100 times greater than the natural concentration in order to achieve a therapeutic effect.

### 5. Safety Studies of Deuterium Oxide

Over 80 years of studies on deuterium oxide have clearly delineated its safety profile for many species, including humans. The non-radioactive deuterium isotope of hydrogen constitutes about 1/6400 of all hydrogen atoms in nature. The natural concentration of deuterium oxide in water is about 150 parts per million (0.01%), and each human already contains about 5 g of heavy water. Generally, the more complex the organism, the more vulnerable it is to increased concentrations of deuterium oxide. One-celled algae can survive long-term in 100% deuterium oxide, and fish and tadpoles can survive long-term in 30% heavy water. The biological effects of deuterium oxide in mammals were first studied in 1937 by Barbour. By 1960, Thompson published a review of the numerous studies of these effects. A review study by Peng in 1972 concluded that a drinking water concentration of 5% deuterium oxide was safe long term for mammals. In mice and rats, deuterium oxide intoxication signs and symptoms generally do not occur until over 15% of body water is replaced by deuterium oxide and maintained long-term. First symptoms were hyperexcitability and increased aggressiveness. In rats and mice, a body water deuterium oxide concentration of 30-35% produces coma and then death. Histologic changes caused by high deuterium oxide concentrations are similar to radiation damage, because deuterium oxide begins to interfere with cell mitosis. Tissues with high mitotic activity are especially vulnerable: hematopoiesis, formation of intestinal mucosa, and spermatogenesis are particularly harmed at high doses. Interestingly, neoplastic cells are more vulnerable than normal cells. High doses and prolonged exposure are toxic for eukaryotes also due to the inhibition of enzyme activity, as bond strength between deuterium and carbon is 10 times stronger compared to that of hydrogen.

Humans can tolerate fairly high levels of deuterium in body fluids. Acute replacements of 15-23% of total body water with deuterium have been reported in cancer therapy treatments with no evident adverse effects. Achieving a 15% deuterium oxide concentration in a 70 kg male containing 50 L of total body water (TBW) would require a dose of 7.5 L deuterium oxide. In 1995 Wallace found that concentrations as high as 23% in human fluids were found not to be toxic over short time periods.

To reach a level of 10% of deuterium oxide in body water, which might or might not be toxic, a 70-kg human (with about 50 L body water) would have to drink 5 L of pure deuterium oxide. Higher doses and prolonged exposure are toxic for eukaryotes due to the inhibition of enzyme activity as bond strength between deuterium and carbon is 10 times stronger compared to that of hydrogen. Total body water concentrations of 15% deuterium oxide or greater is generally considered the toxic threshold for humans. Deuterium oxide has no metabolites, active or inactive.

In a 1950 study, using deuterium oxide as a tracer for studying cholesterol synthesis, London and Rittenberg noted no adverse effects in a subject given 225 g deuterium oxide per day (diluted in a 10% solution) for 2 days. Subsequently, 22.5 g deuterium oxide per day was given for 18 days, again diluted in 10% solution.

In 1966, Taylor and colleagues gave five human subjects 140-250 ml of 100% deuterium oxide with no “noteworthy or abnormal reactions to deuterium oxide,” other than short-term vertigo in two of the subjects. The vertigo began 30 minutes after this 140-250 ml dose and lasted for as long as a few hours. The vertigo was believed to be due to disturbed vestibular function as the slightly more dense and viscous deuterium oxide equilibrated in the endolymph of the inner ear. This loading dose was followed by deuterium oxide administration over 6-8 weeks that maintained their body water at 0.5% deuterium oxide (a 70 kg male with 50 kg total body water would need 250 ml of deuterium oxide to reach a deuterium oxide concentration of 0.5% deuterium oxide). Taylor suggested that the threshold for noticeable side-effects exists at some dosage between 70 g and 140 g of 100% of deuterium oxide, although subjects given the latter dose gradually over the evening period report a lessening or absence of vertigo effects.

In an online report (reddit.com), one person reported consuming one liter of heavy water for a medical research project on T1 helper cells. He developed severe vertigo with emesis, but reported that he felt “OK” after a few hours and was able to go skiing that evening. A few subsequent daily doses of 200 cc had no noticeable adverse effects on him.

In 1977, Given et al. studied the influence of deuterium oxide on the vestibular system. Similar to the more severe side effect of vertigo that had been noted with deuterium oxide doses of 140-250 g, Given observed positional ocular nystagmus when a pure deuterium oxide dose of 25-50 ml or greater was drunk. The nystagmus began about 20 minutes post ingestion and continued for up to 140 minutes. These subjects frequently experienced mild Gl upset and dizziness as well.

In 1979, Coward reviewed seven studies of babies (mostly studies on state of hydration) in which single deuterium oxide doses of up to 2 ml/kg were given babies, with no observed harmful effects (for a 70 kg male, 2 ml/kg would be a single dose of 140 ml deuterium oxide).

In his 1972 review of the effects of long term deuterium oxide exposure, Peng recommended that 1% deuterium oxide in the drinking water be considered a safe long term exposure for humans. Such exposure would result in long term maintenance of approximately a 1% deuterium oxide concentration in the patient’s total body water.

Note that this application refers to a number of publications and other references, sometimes by author(s) and year of publication. Discussion of such publications herein is given for more complete background and is not to be construed as an admission that such publications are prior art for patentability determination purposes.

### BRIEF SUMMARY OF EMBODIMENTS OF THE PRESENT INVENTION

Embodiments of the present invention use deuterium oxide for the more effective treatment of psychiatric or mood disorders, including depression and anxiety, and circadian rhythm sleep disorders in a subject in need thereof, for example a mammal such as a human.

It is also an object of the present invention to provide a rapidly-acting chronobiological treatment of depression by administering deuterium oxide.

It is also an object of the present invention to provide a rapidly-acting chronobiological treatment of anxiety by administering deuterium oxide to a subject in need thereof.

Another object is to provide an antidepressant medication that can be accurately and easily dosed to achieve the desired concentration of antidepressant in each individual patient in need of treatment.

A further object is to provide an antidepressant to a subject in need of treatment with the antidepressant and wherein the concentration of the antidepressant in the blood of a subject treated with the antidepressant and in all bodily aqueous fluids can be conveniently and safely determined by non-invasive procedures.

A further object is to provide an anti-anxiety agent to a subject in need of treatment with the anti-anxiety agent and whose concentration in the blood and in all bodily aqueous fluids can be conveniently and safely determined by non-invasive procedures.

An additional object is to provide a safe treatment of depression to a subject in need of treatment by administering a compound already given in greater than naturally-occurring concentrations without harm to infants, children, nursing mothers and other adults.

An additional object is to provide a safe treatment of anxiety to a subject in need of treatment by administering a compound already given in greater than naturally-occurring concentrations without harm to infants, children, nursing mothers and other adults.

Another object is to use deuterium oxide to change circadian rhythms to treat circadian rhythm sleep disorders.

The foregoing objects can be accomplished by administering deuterium oxide to a patient suffering from depression, anxiety or a circadian rhythm sleep disorder (as those conditions are described in the Fifth Edition of the Diagnostic and Statistical Manual of the American Psychiatric Association). Embodiments of the present invention are directed to methods of treatment by providing a patient an amount of biological grade deuterium oxide sufficient to alleviate the depression, anxiety, or circadian rhythms sleep disorder but insufficient to cause adverse toxic effects, which is drunk, undiluted or mixed in water, by the patient in need of antidepressant, anti-anxiety or circadian rhythm sleep disorder therapy. In one embodiment, the deuterium oxide is taken undiluted in a single dose. In another embodiment, the deuterium oxide is diluted in a single dose. In another embodiment, the deuterium oxide is diluted in divided doses given throughout the day, and is taken daily for a period of time sufficient to treat the symptoms of depression, anxiety or circadian rhythm sleep disorder. Since deuterium oxide is distributed evenly and rapidly throughout the total body water, the concentration of deuterium oxide in the blood in the brain may be determined by measuring the deuterium oxide concentration in any bodily fluid specimen, such as saliva or urine. Subsequent dosage could then be added to achieve a concentration adequate to elicit an antidepressant, anti-anxiety or sleep/wake circadian rhythm adjustment effect while avoiding unwanted effects.

Embodiments of the present invention further involve the administration of deuterium oxide mixed in sufficient parts water to achieve a solution of deuterium oxide that produces at least one of an antidepressant effect, anti-anxiety effect or sleep/wake circadian rhythm adjustment effect while avoiding one or mor of the following unwanted effects, adverse toxic or psychological effects, delayed onset of action or low response rate.

Embodiments of the present invention also involve the administration of deuterium oxide in total cumulative mass amounts sufficient to raise total body water concentrations of deuterium oxide into a range great enough to produce an antidepressant effect, anti-anxiety effect or sleep/wake circadian rhythm adjustment effect, but less than that which causes adverse toxic effects.

Embodiments of the present invention also involve the administration of a solution of scopolamine and deuterium oxide. The scopolamine dissolved in the deuterium oxide that is administered in total cumulative mass amounts sufficient to raise total body water concentrations of deuterium oxide into a range great enough to produce an antidepressant effect, anti-anxiety effect or sleep/wake circadian rhythm adjustment effect, but less than that which causes adverse toxic effects. The dose-limiting deuterium oxide side effect in humans is an acute, brief period of nausea and vertigo (“motion sickness”). Scopolamine in a dosage range FDA-approved to treat “motion sickness” will be given in solution with the deuterium oxide in this embodiment in order to provide prophylaxis against a possible side-effect of “motion sickness” from the deuterium oxide.

Objects, advantages and novel features, and further scope of applicability of the present invention will be set forth in part in the detailed description to follow, and in part will become apparent to those skilled in the art upon examination of the following, or may be learned by practice of the invention. The objects and advantages of the invention may be realized and attained by means of the instrumentalities and combinations particularly pointed out in the appended claims.

### DETAILED DESCRIPTION OF THE INVENTION

As discussed in the background of the present application, mood can be defined as a predisposition for a particular type of activity or inactivity. The first such internally controlled predispositions were circadian rhythms. The symptoms and longitudinal history of Major Depressive Disorder (“MDD”) are intertwined with numerous circadian and circa-annual rhythms. MDD is related to a disruption in the master circadian clock generation and synchronization of circadian rhythms. Currently, a few empirically-validated chronotherapies utilize a zeitgeber such as bright light or early-morning-awakening (and perhaps induction of a generalized seizure) that resets and resynchronizes circadian rhythms controlled by the suprachiasmatic nucleus (“SCN”). Deuterium oxide penetrates the SCN and slows the period of the circadian rhythms generated and synchronized by the SCN.

Embodiments of the present invention are directed to an acute 100-fold elevation of deuterium oxide concentration in the SCN to reset/resynch the disordered circadian rhythms underlying the pathophysiology of MDD. Given the response time for other efficacious chronobiological treatments, it is hypothesized that the antidepressant effect of the deuterium oxide treatment will be rapid--within a day or so of receiving the deuterium oxide.

The theory underlying an embodiment of the present invention is that circadian rhythm abnormalities are involved in the pathophysiology of mood disorder. More speculatively, if mood is viewed as a predisposition for certain types of action or inaction, the evolutionary roots of mood itself are based in the ability of subsystems within the organism to activate or depress readiness for certain types of activity or inactivity. Organisms early on developed subsystems—periodic clocks--to anticipate rhythmically changing conditions on the earth as it spun on its axis and rotated about the sun. Sleep/wake cycles, hormonal and neurotransmitter circadian cycles, hibernation, migration patterns, and seasonal estrous cycles are examples of timing systems which put the organism “in the mood for” the appropriate activity at the appropriate time. Theoretically, altering these timing subsystems will enable one to manipulate mood itself.

Chronobiological theory applied to psychiatric disorders is a major part of this theoretical basis for this invention. A classic book in the field summarizes the theory succinctly: “There are several reasons to consider the role of circadian rhythm disturbances in affective illness. Because of its inherent cyclicity, the illness itself is a kind of abnormal biological rhythm spanning weeks, months, or years. Circadian rhythms are implicated in some of the symptoms of depression, such as early awakening and diurnal variation in mood. The possible importance of the circadian system in its pathogenesis is suggested by the capacity of experimental alterations in the timing of sleep and wakefulness to alter clinical state.” (Circadian Rhythms in Psychiatry, T.A. Wehr and F.K. Goodwin, 1983, pg. 5). Similarly, the circadian rhythm sleep disorders are viewed from the perspective of chronobiological theory since, by definition, they are the subset of sleep disorders involving circadian rhythm disruption.

The next underpinning of this invention is from biological studies which demonstrate that deuterium oxide is able to alter the body’s master clock (located in the brain’s suprachiasmatic nucleus). In general, “the living clock is virtually intractable to exogenous chemical manipulation.” (The Chronomutagenic Effect of Deuterium Oxide on the Period and Entrainment of a Biological Rhythm, H.B. Dowse and J.D. Palmer, Biol. Bulletin 143:513, 1972). Deuterium oxide, however, alters the clock powerfully and reliably in all species studied since 1960.

Another theoretical underpinning of this invention is from chemical studies. The hydrogen isotope deuterium has the same single electron configuration as the protium isotope of hydrogen and therefore enters into essentially the same chemical reactions. With the added mass of an additional neutron, however, the reactions are generally slower (a “kinetic isotope effect”). While deuterium oxide permeates the entire body water, its slowing effect may be particularly significant in the living clock. Other mechanisms of action are possibly also involved in the demonstrated ability of deuterium oxide to alter circadian rhythms. The ability of embodiments of the present invention to treat depression, anxiety and circadian rhythm sleep disorders may also turn out to be due to as yet unknown theoretical factors.

Other scientific fields drawn upon to create this invention are infant nutrition and hydration, pediatrics, human cholesterol synthesis, radiology, and other scientific disciplines where human subjects have safely been given varying amounts of deuterium oxide over short-term and long-term studies since 1949. Research showing it could be given safely to mammals was conducted shortly after the isotope was discovered in 1932.

According to an embodiment of the present invention, a person suffering from the psychiatric disorders depression or anxiety or a circadian rhythm sleep disorder is preferably given a therapeutically effective, non-toxic dose of deuterium oxide. The biological grade deuterium oxide is preferably undiluted or in solution with water. The deuterium oxide is preferably drunk in a single dose to achieve therapeutic effect. It might also be consumed in divided doses, consumed over one or many days. Single doses of less than about 500 grains of deuterium oxide are preferably used. Prophylactic medication to eliminate or mitigate common unpleasant side effects may be administered before or in combination with the dose of deuterium oxide. Dilution of the deuterium oxide in water may be done to reduce the possibility of side effects. The total deuterium oxide amount given is kept below that which causes toxic effect; daily doses less than about 1000 grams, and replacement of less than about 5% of total body water with deuterium oxide would likely be upper safety limits for a short period of time (e.g. less than one week). Exact determination of the patient’s total body water (“Total Body Water” or “TBW”) can be calculated from the bodily fluid sample taken shortly after the first deuterium oxide dose by the general formula: (Dose D20 Concentration) x (Dose Volume) = (Sample D20 Concentration) x (Total Body Water Volume).

In general, the Total Body Water is approximately 65% of a human’s body weight. Since deuterium oxide freely and rapidly enters the total body water, non-invasive testing of urine or saliva can be used to measure for the presence of toxic or therapeutic levels in the blood. Currently available mass spectrometry, infrared spectrophotometry, or other methods can be used to determine the deuterium oxide concentration in the body fluid. The deuterium oxide is preferably given in a large enough dose and for long enough time to alleviate the depression.

Preferably, a total quantity of deuterium oxide equal to approximately 1 % of the patient’s Total Body Water (TBW) would be given. For example, a 70 kg patient with 45 L of TBWwould be given a total of 450 g of deuterium oxide to achieve a 1% concentration of deuterium oxide in the TBW (450 g deuterium oxide /45,000 g water). This deuterium oxide dosage would preferably be given without further dilution in water. The dosage would preferably be preceded by prophylactic use of appropriate medication to eliminate or mitigate unpleasant side effects often associated with ingestion of deuterium oxide. The deuterium oxide would preferably be given until the depression is alleviated, and could also be continued subsequently for a longer therapeutic period as well. The deuterium oxide could also be combined with other treatment modalities for depression, such as other antidepressant medications, sleep deprivation therapy, light therapy, or other psychiatric medications, or other treatments used for depression, to improve their response rate, reduce their time of onset of action, allow the use of lower dosages or duration of treatment, or improve their ability to treat depression in other ways.

As discussed above, the total dosage of deuterium oxide, solutions thereof, pharmaceutical compositions made therefrom and combination therapies that incorporate deuterium oxide and treatments using the same vary depending on the intended effect, characteristics of the patient (e.g., weight, age, sex), the severity of the patient’s condition and the sensitivity of the patient to any side effects of the deuterium oxide. Preferably, the total dosage of deuterium oxide over a given amount of time ranges from about 0.1% of total body water to about 15% of total body water, more preferably about 0.5% of total body water to about 10% of total body water, and most preferably about 1 % total body water to about 5% total body water.

In another embodiment of the present invention, the concentration of deuterium oxide in water consumed by the patient is from about 0.05% to about 100%. In another embodiment, the concentration of deuterium oxide in water consumed by the patient is from about 0.1% to about 15%.

In another embodiment of the present invention, daily amount of deuterium oxide administered is from about 1 gram to about 1500 grams. In another embodiment, the daily amount of deuterium oxide administered is from about 150 grams to about 500 grams.

A person suffering from a circadian rhythm sleep disorder, such as jet lag type or shift work type, would be given a therapeutically effective, non-toxic dose of deuterium oxide to treat this disorder.

Embodiments of the present invention comprise various mediums of delivering the deuterium oxide. Preferably, the deuterium oxide is delivered orally in liquid form. In some embodiments, the deuterium oxide is delivered in combination with other potable liquids in order to reduce side effects or to improve tolerability.

In summary, deuterium oxide, an established potent chronomutagenic agent, will be administered to provide a more effective treatment for depression, anxiety or circadian rhythm sleep disorders, yielding an effective, rapidly-acting, safe, non-addicting medication which can be precisely dosed and easily measured in the patient with non-invasive procedures.

### Additional Examples of Compositions and Methods

Additional embodiments of the present invention are discussed below.

Beginning with concerns for safety, a deuterium oxide dosage sufficient to achieve a 1 % concentration of deuterium oxide in Total Body Water is given. In order to eliminate or mitigate a possible unpleasant side effect of dizziness, nausea, or vertigo, a prophylactic dose of scopolamine is combined into the administered therapeutic mixture. As noted above, the dose-limiting side effect for deuterium oxide would most likely be this unpleasant “motion sickness” side effect. As an example of dosage amounts, a 70 kg male with 65% total body water would have a Total Body Water estimate of 45 L, and would be given 450 g of 99+% biological grade deuterium oxide to drink. This dose would result in about a 1 % concentration of deuterium oxide in the patient’s total body water. This dose of about 0.5 L, about 2 cups, is preferably taken orally (by drinking) in one dose. If needed, the dose could be diluted and/or split into a three-times-a-day regimen for improved tolerability. The dosage of scopolamine in the 0.5 L therapeutic mixture is preferably about 0.3-0.6 mg. The usual half-life of scopolamine is 8 hours. It is anticipated that deuterating the scopolamine will likely prolong this half-life. Preferably, the scopolamine would be deuterated, or might occur naturally if the scopolamine remains in the deuterium oxide mixture at room temperature for a sufficient time.

The estimated Therapeutic Index (ratio of toxic dose to therapeutic dose) of this acute treatment is conservatively estimated at about 15. A deuterium oxide dose that replaces 15% or more of TBW with deuterium oxide over a long term is toxic; a dose achieving 1 % total body water concentration of deuterium oxide is deemed safe over the long-term. The proposed experiment would involve short term exposure to deuterium oxide. Deuterium oxide is excreted by the kidneys with an elimination half-life of up to 10 days. Any bodily fluid (saliva, urine, blood) can be sampled to track the deuterium oxide concentration at which therapeutic benefit and/or adverse events occur. Such ease of monitoring is not available for any other psychiatric medication. Since pure heavy water tastes slightly sweet, a small amount of sweetener is preferably added to the placebo natural water.

Preferably, the treatment would then follow the bedtime dose of the deuterium oxide/scopolamine solution with early (3:00am) awakening of the patient, to achieve a synergistic effect with the proven short-term antidepressant effect of Wake Therapy chronobiological treatment.

The subjects would be diagnosed with Major Depressive Disorder. No particular exclusions would be needed. Severity of depression would be measured by a standard instrument, such as Ham-D. Ideally the rating instrument would be sensitive to short-term, daily change. Depression status would be monitored each day for a few days after deuterium oxide treatment. If effective, the described treatment regimen is predicted to take effect by day 2.

Embodiments of the present invention can include every combination of features that are disclosed herein independently from each other. Although the invention has been described in detail with particular reference to the disclosed embodiments, other embodiments can achieve the same results. Variations and modifications of the present invention will be obvious to those skilled in the art and it is intended to cover all such modifications and equivalents. The entire disclosures of all references, applications, patents, and publications cited above and/or in the attachments, and of the corresponding application(s), are hereby incorporated by reference. Unless specifically stated as being “essential” above, none of the various components or the interrelationship thereof are essential to the operation of the invention. Rather, desirable results can be achieved by substituting various components and and/or reconfiguration of their relationships with one another. The various embodiments of the present invention described herein as methods should also be interpreted to describe compositions. For example, a method of administering deuterium oxide also inherently describes a composition comprising the particular dose of deuterium oxide described by the method.

Note that in the specification and claims, “about” or “approximately” means within twenty percent (20%) of the numerical amount cited. As used herein “a”, “the” and “an” means one or more unless the context clearly indicates otherwise.

A “patient” or “subject,” as used herein, is intended to include either a human or non-human animal, preferably a mammal, e.g., a monkey. Most preferably, the subject or patient is a human.

What is claimed is:

1. A method for treating psychiatric disorders, the method comprising administering a therapeutically effective non-toxic dose of deuterium oxide to a patient suffering from a psychiatric disorder.

2. The method of claim 1, wherein the psychiatric disorders are selected from depression, anxiety and circadian rhythm sleep disorder.

3. The method of claim 1, wherein the concentration of deuterium oxide in water consumed by the patient is from about 0.05% to about 100%.

4. The method of claim 1, wherein the concentration of deuterium oxide in water consumed by the patient is from about 0.1 % to about 15%.

5. The method of claim 1, wherein the total dosage in grams of deuterium oxide provided to the patient is about 0.5% by weight of the patient’s total body water to about 10% of the patient’s total body water.

6. The method of claim 1, wherein the total dosage in grams of deuterium oxide provided to the patient is about 1% by weight of the patient’s total body water to about 5% of the patient’s total body water.

7. The method of claim 1, wherein the daily amount of deuterium oxide administered is from about 1 gram to about 1500 grams.

8. The method of claim 1, wherein the daily amount of deuterium oxide administered is from about 150 grams to about 500 grams.

9. The method of claim 1, further comprising administering the dose of deuterium oxide in a single dose and administering it with a dose of scopolamine.

10. A composition for treating psychiatric disorders comprising a therapeutically effective non-toxic dose of deuterium oxide.

11. The composition of claim 10, wherein the psychiatric disorders are selected from depression, anxiety and circadian rhythm sleep disorder.

12. The composition of claim 10, wherein the total dose of deuterium oxide is from about 1 gram to about 1500 grams.

13. The composition of claim 10, wherein the total dose of deuterium oxide is from about 150 grams to about 500 grams.

14. The composition of claim 10, further comprising scopolamine.

15. The composition of claim 10, wherein the dose of deuterium oxide is about 500 ml in solution with about 0.3 mg to about 0.6 mg of scopolamine.

16. A method of manufacturing a composition for the treatment of psychiatric disorders, the method comprising diluting a dose of deuterium oxide in water.

17. The method of claim 16, wherein the psychiatric disorders are selected from depression, anxiety and circadian rhythm sleep disorder.

18. The method of claim 16, wherein the dose of deuterium oxide is from about 1 gram to about 1500 grams.

19. The method of claim 16, wherein the dose of deuterium oxide is from about 150 grams to about 500 grams.

20. The method of claim 16, further comprising mixing scopolamine with the diluted deuterium oxide.
